(19) United States
(12) Patent Application Publication
(10) Pub. No.: US 2025/0058018 A1
(43) Pub. Date: Feb. 20, 2025

(54) IMPLANTS WITH BIOACTIVE COATING COMPRISING PARTIALLY DEACETYLATED CHITOSAN

(71) Applicant: Genis HF., Siglufjordur (IS)

(72) Inventors: Chuen How NG, Gardabaer (IS); Gissur ORLYGSSON, Mosfellsbaer (IS)

(21) Appl. No.: 18/721,733

(22) PCT Filed: Dec. 20, 2022
(86) PCT No.: PCT/US2022/050009

§ 371 Date: Jun. 19, 2024

(30) Foreign Application Priority Data
Dec. 20, 2021 (IS) 050351

(51) Int. Cl. A61L 27/20

(52) U.S. Cl. CPC A61L 27/20

(57) ABSTRACT

Provided are compositions useful for coating implants, comprising microparticles containing partially deacetylated chitosan with a degree of deacetylation in the range of about 30% to about 75%, with the microparticles generally having an average particle diameter that is less than 50 μm. Also provided are surgical implants containing a coating non-crosslinked partially deacetylated chitosan (PDC) having a degree of deacetylation in the range of about 30-75%, and methods of coating such implants.

### FIELD

The invention is within the field of medical implants such as implants used in orthopaedic applications, and relates to chitosan-based coatings suitable for such implants, coated implants and methods for the preparation of coating compositions.

### INTRODUCTION

By current industrial definition, chitosan refers to highly deacetylated chitin, usually higher than 70% degree of deacetylation (or 70% DD), and will dissolve in weak acids. Chitin refers to low deacetylated chitonous material, generally less than 25% DD, and will not be dissolved in weak acids. The class in between chitin and chitosan is named partially deacetylated chitin (PDC). Industrial usage of PDC is currently uncommon, due to its poor solubility and technical difficulty in large-scale production.

Titanium implants are well known and generally considered the gold standard in load-bearing orthopaedic applications. The advantages of titanium and its alloys for biomedical implants and devices include (i) biocompatability, owing to spontaneous formation of oxide layers, (ii) high corrosion resistance, (iii) high specific strength and (iv) absence of toxicity. Osseo-integration is the factor that has an important effect on the lifetime of the implant and the success of its integration. The term refers to the direct structural and functional connection between living bone and the surface of a load-bearing artificial implant. Osseo-integration is observed with conventional titanium implants but can be compromised by the local conditions and interaction of the implant with the surrounding site of implantation. Various coating materials have been considered and tested to improve the characteristics of implants the interaction of the implant with tissue. Thus, bioactive materials are desired that enhance bioactivity, osseo-integration and implant stabilisation. One of the materials that have been tested is chitosan. The prerequisite for the preparation of chitosan coatings is the cationic nature of chitosan, as it is the premise for the solubility in dilute aqueous acids. Li et al. (Biomaterials 36 (2015) 44-54) describe chitosan-coated porous titanium alloy implants implanted in diabetic sheep, where the coated implants ameliorate overproduction of ROS (reactive oxygen species) that is believed to cause poor osseo-integration of implants in diabetic implant patients. Chitosan with 85-90% degree of deacetylation (DDA) was used. Husain et al. (Materials (2017) 10, 602) discusses chitosan-coated dental implants and cites earlier studies have reported promising results for chitosan coating of dental implants and discloses that the chitosan coating may affect the surface and bone interface by altering biological, mechanical and morphological properties. The review however submits that further research was required to validate if such coatings are beneficial to inhibit infection and promote osseointegration.

### SUMMARY

The present invention provides new and improved compositions for coating implants. The compositions contain an amorphous partially deacetylated chitosan (PDC) that is soluble in an acidic aqueous solution and has a high swelling capability.

The PDC can be prepared by dissolution of partically deacetylated chitin in acid and purification through serial filtration, rendering a resulting regenerated PDC that is completely solubilizable in an acidic aqueous solution and that has a high swelling capability in an aqueous solution. Thus, the PDC can absorb as much as 10×, 15×, or 20× or more of its weight of water.

The compositions containing the PDC can be used as coatings on e.g. surgical implants. Accordingly, also provided are surgical implants coated with the coatings of the invention. Additionally, this coating may incorporate with active ingredients or cells to improve the new bone formation. For example, the coating can contain growth factors, growth-promoting or growth-enhancing drugs or other substances that are beneficial for bone formation. The coating can also contain antibacterial and/or antiviral compounds such as antibiotics.

In an aspect, there is provided a chitosan-based coating for a surgical implant, the coating comprising non-crosslinked partially deacetylated chitosan (PDC) having a degree of deacetylation in the range of about 30-75%. In exemplary embodiments, the degree of deacetylation can be in the range of about 35% to about 70%, in the range of about 35% to about 60%, in the range of about 40% to about 75%, in the range of about 40% to about 60%, or in the range of about 45% to about 55%. It can be beneficial to have a degree of deacetylation near or at 50%, such as about 46%-54%, 47%-53%, 48%-52%, or 49%-51%. The disclosure also relates to implants that have at least a part of the surface coated with a PDC-based coating, in particular, a PDC-based coating as disclosed further herein.

The disclosure also relates to methods for coating of implants with PDC, including (i) coating of a fully dissolved PDC, i.e. PDC in form of true solution, and (ii) non-homogenous or physically dispersed PDC, i.e. PDC that is not dissolved but provides a coating in colloidal form.

In the present context, the term “true solution”, refers to a homogeneous mixture (solution) of two or more components, where the particle size of the dissolved component (solute) is less than 220 nm. By way of example, a simple sugar solution in water is a true solution.

In the present context, the terms “dry thickness”, sometimes also referred to as “dry film thickness”, and “wet thickness”, sometimes referred to as “wet film thickness”, refer to the thickness of a film or coating on a surface after and before drying, respectively. The two parameters are related by the volume fraction of solids in the film or coating, i.e.: (dry thickness)=(wet thickness)×% (volume solids).

A “homogenous coating”, as described herein, refers to a coating that covers substantially the entire underlying surface, preferably 100% of the underlying surface. By contrast, a “non-homogeneous coating”, as described herein, refers to a coating that covers less than 100% of the underlying surface, i.e. parts of the underlying surface are not covered by the coating.

The PDC may be in the form of microparticles. The microparticles may have generally spherical shape. The spherical shape may be generally round, or it can be oblong such as in the shape of a prolate spheroid. The microparticles can also have a polyhedron shape, i.e. have a shape that is three-dimensional with generally flat polygonal surfaces. The microparticles may alternatively have an irregular shape. The microparticles can be homogeneous in shape (i.e. a population of particles is generally identical in shape), with varying dimensions (size). Alternatively, the microparticles can be heterogeneous in shape and/or dimensions. For example, the microparticles can be partially spherical and partially irregular in shape.

Also provided is a composition comprising microparticles containing partially deacetylated chitosan with a degree of deacetylation in the range of about 30% to about 75%, the microparticles having an average particle diameter that is less than 50 μm. The composition can have a degree of deacylation in the range of about 35% to about 70%, in the range of about 35% to about 60%, in the range of about 40% to about 75%, in the range of about 40% to about 60%, or in the range of about 45% to about 55%.

The particle size can be less than 40 μm, less than 30 μm, or less than 20 μm, such as in the range of about 5 to 50 μm, in the range of about 5 to 40 μm, in the range of about 5 to 30 μm, in the range of about 5 to 25 μm, in the range of about 5 to 20 μm, or in the range of about 5 to 15 μm.

The coating composition can preferably be in the form colloidal suspension in an aqueous solution, such as water or a physiological body fluid. Microparticles comprising the PDC can be suspended or physically dispersed in the aqueous solution to obtain a colloidal gel-like matrix comprising the microparticles suspended in the matrix.

Also provided is a surgical implant or part thereof, characterised in that it is coated on its outer surface or a part thereof with a surface coating comprising non-crosslinked partially deacetylated chitosan (PDC) having a degree of deacetylation in the range of about 30-75%.

The term “part thereof” can be understood as one or more component of an implant that comprises multiple (two or more) components. For example, the surgical implant may comprise two or more components, wherein one or more of the components comprise a surface coating in accordance with the invention. An implant can thus be assembled from two or more such components, wherein some, or all, of the components comprise a coating as disclosed herein.

The coating can have a wet thickness (i.e. containing water, before or in the absence of subsequent drying to remove water) in the range of 20 to 250 μm, in the range of 50 to 250 μm, in the range of 30 to 150 μm or in the range of 40 to 100 μm.

The density of N-acetylglucosamine (NAG) on the surface of the implant, or part thereof (i.e. in the coating), can be in range of 0.01-15 mg/cm², or 0.3-5 mg/cm², preferably 0.3-1.5 mg/cm².

The coating can be non-homogeneous, coating about 10 to about 90% and preferably in the range of about 25% to about 75%, of the outer surface of a unit area of the implant, or part thereof. Alternatively, the coating can be a non-homogeneous coating with a coverage in a unit area (e.g. of 1-5 mm²) that is in the range from 10%-90% and preferably in the range of about 25% to about 75%.

The coating can have a certain roughness, i.e. an uneven structure on its surface, such that some parts of the coating have a larger thickness than other parts of the coating. Such coating can be described as having peaks and valleys, with peaks corresponding to regions with relatively large thickness, and valleys corresponding to regions with relatively small thickness.

The chitosan-based coating can have a dry surface roughness, i.e. roughness following drying of the coating so that the coating is in a dry state, that is less than 500 μm, such as in the range of about 2-500 μm, in the range of 5-250, in the range of about 20-100 μm, in the range of about 25-100 μm, in the range of about 5-100 μm or in the range of 5-50 μm. The roughness can be defined as the difference between the greatest and smallest thickness of the coating on the surface.

Also provided is a method of coating a surface of a substrate, e.g. a surgical implant, with non-crosslinked partially deacetylated chitosan (PDC) having a degree of deacetylation (DD) in the range of about 30-75%, the method comprising immersing the substrate surface in an aqueous solution comprising such partially deacetylated PDC, removing the surface from the solution and allowing to dry.

Additionally, the homogenous and the non-homogenous coating may be integrated. For example, the substrate can be homogenously coated with one layer of PDC, follow by a layer of non-homogenous coating to provide a rougher surface for better cell adhesion and proliferation.

The above features along with additional details of the inventions, are described further in the examples below, which are intended to further illustrate the invention but are not intended to limit its scope in any way.

### BRIEF DESCRIPTION OF THE DRAWINGS

The skilled person will understand that the drawings, described below, are for illustration purposes only. The drawings are not intended to limit the scope of the present teaching in any way.

FIG. 1 shows the physical properties of partially deacetylated PDC. (Top) Powdery PDC; (Bottom) 1000× magnification of the PDC obtained by scanning electron microscopy (SEM).

FIG. 2 shows in (a) a coated titanium plate; (b) coated plate with half of the plate gold-coated; (c) height difference (Solarius Profilometer) between the gold-coated area and the remaining area representing the thickness of PDC coating.

FIG. 3 shows in (a) a 3D view of the surface roughness of a typical titanium plate, coated with 1% PDC (w/v); (b) topography image of the coated surface in 2D view; (c) Actual recorded surface roughness along the line as shown in FIG. 3(a); (d) distribution of surface roughness along the line as shown in FIG. 3(a).

FIG. 4 shows the impacts of the swelling properties of PDC films when different fluids were used for preparing the PDC solution. (a) with water only as fluid and (b) with ethanol:water (1:1) as fluid.

FIG. 5 shows the relationship of the swelling index of PDC films, before and after 4-week of soaking in water, and the temperature used to dry the film.

FIG. 6 shows Scanning Electrode Microscopy (SEM) images of titanium plate coated with PDC dissolved in acetic acid. (a) 2000× magnification, (b) 100,000× magnification.

FIG. 7 shows PDC coating on a titanium substrate. (a) coating obtained by soaking in a fully dissolved PDC solution (true solution); (b) coating obtained by soaking in colloidal PDC.

FIG. 8 shows PDC coating on a bioglass substrate. (a) coating obtained by soaking in a fully dissolved PDC solution (true solution); (b) coating obtained by soaking in colloidal PDC. (Top right) Surface coated by the colloidal method produces an organic structure and facilitates better cell attachment.

FIG. 9 shows PDC coating on a hydroxyapatite substrate. (a) coating obtained by soaking in a fully dissolved PDC solution (true solution); (b) coating obtained by soaking in colloidal PDC.

FIG. 10 shows material loss after prolonged soaking in water.

FIG. 11 shows surface distribution by SEM upon treatment with PDC in PBS buffer. (a) 200×, and (b) 2500× magnification.

FIG. 12 shows Atomic Force Microscopy (AFM) images of PDC coating obtained by (a) fully dissolving chitsoan in acid and coating onto titanium by homogeneous method; and (b) PDC dispersing PDC in PBS solution and coating onto titanium by the non-homogeneous method.

### DESCRIPTION

The present invention sets forth a highly stable bioactive surface coating comprising non-crosslinked partially deacetylated chitosan, PDC. The coating can be performed either in homogeneous or non-homogenous manner, i.e. covering substantially the entire surface or only partially covering the substrate.

The PDC can preferably be prepared by a regenerative process, whereby chitosan, prepared by partial deacetylation of chitin material, is first dissolved in acid and subsequently purified by filtration and reprecipitated from the acidic solution.

Chitosan is prepared by deacetylation of chitin. Thus, chitin can be deacetylated in concentrated sodium hydroxide to deacetylate the chitin to an appropriate extent, in the present case generally providing 30-75% deacetylated material. Deacetylation can be performed at relatively low temperature, such as in the range of about 20-60° C., such as 30-60° C., 40-60° C., 45-55° C. or about 50° C., to minimize the formation of a blocked distribution of N-acetylglucosamine (NAG) in the resulting chitosan material and thereby minimize the crystalline properties of the obtained material. More clustered NAG will increase the chain to chain interactions, this interferes the chain separation upon protonation of the polymers, and thus the solubility of the material. Moreover, clustered NAG will increase the interactions forces among chains upon drying of the material, resulting in a less amorphous material.

Deacetylation is followed by the removal of the alkali by washing and the recovery of the chitosan material. Then the chitosan is dissolved in acid to facilitate the removal of all insoluble materials in the resulting fluid, for example by one or more steps of filtration (e.g. coarse filtration followed by ultrafiltration). After that the solution is subsequently neutralized using an appropriate base such as NaOH and salt, and the chitosan is precipitated from the solution to obtain the pure PDC. The PDC can be optionally dried, for example by spray-drying, to obtain a dry, white microparticle PDC material. An example of the present process is described in Example 1 herein.

The resulting PDC has certain distinctive properties that make it particularly useful for the present purpose.

Thus, the reprecipitated (regenerated) PDC is generally amorphous (i.e., not crystalline) in nature and consists of uniform, or near-uniform, fine microparticles. The particles are usually less than 50 μm in diameter, and can be generally spherical in shape.

Classical chitosan is by contrast crystalline and usually very difficult to dissolve. Thus, the dissolution of solid chitosan to obtain a chitosan solution can take many hours or may even be performed overnight. By contrast, the dissolution of the PDC obtained by the present regenerative method is almost instantaneous. For example, upon dispersing the PDC in a fluid, e.g. in water, the addition of suitable acid will transform the colloidal fluid into a crystal clear/homogenous solution within seconds. This PDC solution is, as well more resistant to precipitation around physiological pH and beyond than classical chitosan.

The PDC can have a near to equal ratio of NAG and GluN, or a more balanced hydrophobic/hydrophilic properties. As a consequence, the PDC is able to form a stable mixture with either water-based or most oil-based fluids easily. Moreover, the amorphous and fine particulate nature of the dry PDC enables it to absorb more than 20× of pure water to its weight.

The homogeneous coatings disclosed herein attach firmly to the substrate surface with up to 100% coverage attained, i.e. the coating covering up to 100% of the surface, with no signs of imperfect coating, and methods to control the thickness of the coated film. The coatings show good stability, remaining firmly attached after 4 weeks of soaking in water, with no sign of film detachment or flouting sign sighted.

However, in other useful embodiments, a non-homogeneous coating is provided, and methods for producing such coatings in a controlled manner are provided. The term “non-homogeneous coating” as used herein refers to a coating that covers less than 99% of the underlying surface, such that the coated surface has interspersed areas that are coated and also areas that are non-coated.

Conditions in the coating process can advantageously be adjusted so as to achieve different coverage and different spatial coarseness of the coating to meet the requirement of intended purposes. The non-homogeneity can be determined by analysing a unit area. Such unit area can have a coating coverage in the range from about 10% to about 95%, such as in the range from about 25% to about 75%, and more generally in a range from about 10% or about 15% or about 20% or about 25% or about 30% or about 35%, to about 95% or to about 90% or to about 85% or to about 80% or to about 75%.

The unit area can have any suitable dimensions, for example, defined as a rectangle, square, circle, oval or other regular or irregular shapes. In some embodiments, the unit is a rectangle with each side having a length of 10-20 mm, such as 10-15 mm or about 10 mm.

The dimensions of the unit are tested for homogeneity will represent a characteristic of the overall coating. Thus, when it is desired to obtain coatings with a less coarse, more refine-grained non-homogeneity, a smaller unit area can be selected to determine if the desired non-homogeneity is observed on a smaller scale, such as rectangular unit area of less than 10×10 mm, such as 5×5 mm, 2×2 mm or 1×1 mm, with the desired coating coverage in the area within any of the above-mentioned coverage ranges.

The thickness of the coating after immersing in an aqueous or PBS solution is adjustable. The coating with the homogeneous method will generally swell to a thickness 2-10 times, or 5 to 15 times, or more typically 6-12 times to its thickness at dry. For the non-homogenous method, the thickness of the coating may range from 5-20 times, or 10-25 times or typically 10 to 20 times to its dry thickness.

The initial thickness of the coating can be influenced by the drying temperature and types of aqueous solvent or fluid used for the preparation of the PDC solution. The coating may be dried by treatment in a temperature range from −40° C. to 100° C. or 0-80° C. or more typically 15-75° C. However, if any appropriate solvent, e.g. an alcohol such as ethanol or any other suitable primary alcohol or mixtures thereof, is/are introduced into the aqueous solution used for dissolution of the PDC, the impact of drying temperature on the initial thickness of the swell film is more limited.

The fluid for preparing the PDC solution is preferably a water-miscible solvent that is mixed with an aqueous solution in a range of solvent:water ratio that is between 1:10 and 2:1, between 1:5 and 2:1, between 1:4 and 2:1, between 1:3 and 2:1 or between 1:2 and 2:1. The fluid, e.g. with water only or mixture of water/alcohol(s), used for preparing the PDC solution can have impacts on the swelling properties and the drying rate of the film as well as a protective measure against microbe contamination during the drying stage.

It is also possible to provide two or more layers of coating on the surgical implants. The coatings can be similar or identical in nature (i.e., homogeneous or non-homogeneous) or the coatings can be different. For example, there can be two (or more) coating steps forming two (or more) coating layers, wherein the first coating step provides a homogeneous coating on the surface, and wherein the first coating step is followed by a second or more coating step(s) that are non-homogeneous in nature. This way, the surface of the implant can be essentially completely coated, with a first homogeneous layer providing a smooth complete coating of the surface, and a second or more subsequent coatings providing a non-homogeneous coating that can be provided by a colloidal coating suspension. This way, there can be a roughness or irregularity of the overall coating, while at the same time ensuring that the entire surface of the implant be covered by at least one coating layer.

The PDC in the coatings has a DD in the range from about 30% to about 75% and preferably in the range from about 35% to about 60%, such as about 50%. The coating has in some embodiments a thickness after drying in the range from about 2 to about 25 μm, or more generally in a range from about 2 μm or from about 4 μm or from about 5 μm or from about 10 μm, to about 25 μm or to about 20 μm or to about 15 μm. Pre-drying thickness can be in the range of 10 to 50 μm or from 10 to 250 μm, where a thicker pre-drying thickness will lead to a thicker dry-state thickness. Upon implantation, the PDC will swell into predesigned thickness to accommodate the interfaces between the implant and the body surfaces.

The coating density of the coating or film may be expressed by the amount of N-acetyl-D-glucosamine (NAG) per unit area, i.e. mg NAG/cm². The typical NAG coating density may range from 0.01 to 15 mg/cm², or 0.3 to 5 mg mg/cm², or more preferably at the range of 0.3 to 1.5 mg/cm².

The surface coating compositions disclosed herein can be used to coat any suitable surgical implant, such as implants comprising, or consisting of metals or alloys, e.g. stainless steel, titanium, titanium alloy, cobalt chrome alloy, bioglass, hydroxyapatite/calcium phosphates or mixtures thereof. The implants can be pre-treated before coating, i.e. some form of pre-treatment may be beneficial for certain applications. The skilled person will however appreciate that pretreatment of the surface is not essential to carry out the invention. Accordingly, in some embodiments the surface of the implant to be coated has been pre-treated prior to coating, such as through oxidation by acid treatment or by washing with alkali solution.

Surgical implants are in general any medical device that is produced to replace a missing biological structure, support a damaged biological structure or enhance the function or structure of an existing biological structure. The implant can be a neurological or sensing implant, a cardiovascular implant, an orthopaedic implant that alleviates issues with bones and/or joints in the body, an electric implant, a contraceptive implant or a cosmetic implant. The implant can be permanent or temporary. In certain embodiments, the implant is a dental implant.

The PDC coatings disclosed herein, and compositions for use in generating such coating, are compatible for use with any such implant, or a portion thereof. The implant can comprise titanium or titanium alloy, cobalt chrome alloy, stainless steel, tantalum, hydroxyapatite/calcium phosphate or bioglass surfaces. The implant can also consist of, or be comprised of, a mixture of any two or more of the foregoing materials, or combinations with other materials that are commonly used in prosthesis or other implants.

The lower end of the DD range can be about 30% or about 33% or about 35% or about 40%. The upper end of the DD range can be about 65% or about 60% or about 58% or about 55%, or about 50% or about 45%. Accordingly, the DD range can be about 30% to 70%, about 35% to about 65%, about 40% to about 60%, about 45% to about 60%, about 45% to about 55%.

Within this DD range the PDC can be effectively substantially dissolved, providing a homogeneous concentration of the PDC and a homogeneous coating of the treated surface. The immersion (coating) solution in certain embodiments preferably has a pH above about 6 and more preferably above about 7, such as in the range from about 7.0 or from about 7.2, to about 7.5, such as about 7.4. The coating solution can also be a non-acidified solution with a pH above 6, preferably a pH in the range of 7-8, more preferable a pH in the range of 7.3-7.5, most preferably a pH of about 7.4.

In other embodiments the immersion solution is acidic, such as having a pH in a range from about 2 to about 4.5. Such acidic immersion solution can for example be acidified with acetic acid in a concentration in the range from about 0.5% to about 2% or 0.1 to 10%, such as about 1%.

The PDC is advantageously provided in the immersion solution in a concentration in a range from about 0.5% to about 1.0%, such as but not limited to a concentration of about 0.5% or about 0.75% or about 1-20%.

Immersion time can range from minutes to days. Thus the immersion time can generally range from 1 minute to several days, such as 5 days. In certain embodiments the immersion time is relatively short, or in the range of about 1 to about 60 minutes, such as about 1 to about 30 minutes, such as about 1 to about 10 minutes, or about 1 to about 5 minutes. In certain embodiments, the immersion time is relatively long, or about 1 hour to 5 days, such as about 2 hours to 2 days, such as 2 hours to 1 day, such as 2 hours to 12 hours, or 2 hours to 6 hours.

The implant surface is suitably dried after the immersion has been completed for a sufficient desired time to allow solvent to evaporate from the surface. The drying can be performed at a temperature that is generally in the range of 0 to 100° C., such as in the range of 10° C. to 100° C., in the range of 20° C. to 100° C., in the range of 20° C. to 90° C., or in the range of 20° C. to 80° C.

The PDC with the above described degree of deacetylation and having a preferred particle size as described herein below can be solubilized in aqueous solution, such as water to obtain a gel-like colloidal matrix that comprises micro-size PDC particles suspended in the gel matrix. The gel matrix thus obtained can be used as the immersion solution in the coating process, producing a desired high-strength coating of the invention.

For the micro-particle coating the particle size of the PDC particles is typically less than 50 μm and preferably less than 30 μm and more preferably less than 20 μm and yet more preferably less than 10 μm, meaning that >95% of the particles have a widest cross-section less than the given numerical figure (e.g., >95% of the particles have a widest cross-section less than 30 μm, or 20 μm, or 10 μm).

Thus, in some embodiments the PDC particles have a particle size distribution in the range from about 1 μm or from about 2 μm or from about 3 μm or from about 4 μm or from about 5 μm to about 20 μm or to about 15 μm or to about 12 μm or to about 10 μm, such as having an average (mean) or median largest diameter of about 5 μm or about 8 μm or about 10 μm or about 12 μm or about 15 μm.

In some embodiments the PDC particles have a mean particle size that is less than about 50 μm, and preferably less than about 20 μm, more preferably less than about 10 μm.

The concentration of the PDC in the immersion gel matrix can be for example in the range from 0.2 wt % to about 20 wt %, and preferably in the range from about 0.5% to about 10%, such as from about 0.5% or from about 1% or from about 2% to about 10% or to about 5% or to about 4%.

It can be beneficial to pre-treat the implant to be coated to increase its adhesive properties and/or its biocompatibility. Pre-treatment can be performed by treatment with an oxidating agent, such as an acid or acid mixture (e.g., sulfuric acid, hydrofluoric acid, phosphoric acid), or hydrogen peroxide, or mixtures thereof. Pre-treatment can alternatively be performed by treatment with an alkali solution, such as sodium hydroxide. Other suitable pre-treatment methods are known in the art, and are also contemplated with the coating described herein.

Exemplary embodiments of the invention include the following:
1. A chitosan-based coating for a surgical implant, the coating comprising non-crosslinked partially deacetylated chitosan (PDC) having a degree of deacetylation in the range of about 30-75%.
2. The chitosan-based coating according to item 1, wherein the degree of deacetylation is in the range of about 35% to about 70%, in the range of about 35% to about 60%, in the range of about 40% to about 75%, in the range of about 40% to about 60%, or in the range of about 45% to about 55%.
3. The chitosan-based coating according to item 1 or item 2, wherein the degree of deacetylation is about 50%.
4. The chitosan-based coating according to any one of the preceding items, wherein the coating has a dry thickness in the range of 2-25 μm, preferably in the range of 5-15 μm.
5. The chitosan-based coating according to any one of the preceding items, wherein the coating has a wet thickness in the range of 20 to 250 μm, preferably in the range of 40 to 100 μm.
6. The chitosan-based coating according to any one of the preceding items, wherein the coating has a surface roughness that is less than 500 μm.
7. The chitosan-based coating according to any one of the preceding items, wherein the coating has a surface roughness that is in the range of about 25-500 μm, in the range of 5-250 μm, in the range of about 20-100 μm, in the range of about 25-100 μm, in the range of about 5-100 μm or in the range of 5-50 μm.
8. The chitosan-based coating according to any one of the preceding items, wherein the coating coats a surface along the topography of the surface to which it is applied to facilitate cell attachment or cell growth on coating.
9. The chitosan-based coating according to any one of the preceding items, wherein the coating comprises a non-homogeneous layer with a coverage over a unit area on a surgical implant that is in the range of about 10 to about 90% and preferably in the range of about 25% to about 75%.
10. The chitosan-based coating according to the preceding item, wherein the unit area is in the range of 25-100 mm², such as in the range of 35-100 mm², such as in the range of 50-100 mm².
11. The chitosan-based coating according to item 10, wherein the unit area is in the range of 1-25 mm², such as in the range of 5-25 mm², such as in the range of 10-25 mm².
12. The chitosan-based coating according to any one of the preceding items, wherein the coating comprises PDC in the form of microparticles with an average particle size of less than 50 μm, less than 30 μm or less than 10 μm.
13. The chitosan-based coating according to any one of the previous items, wherein the microparticle PDC has a mean particle size less than about 50 μm, and preferably less than about 20 μm, more preferably less than about 10 μm.
14. A composition comprising microparticles containing partially deacetylated chitosan with a degree of deacetylation in the range of about 35% to about 70%, in the range of about 35% to about 60%, in the range of about 40% to about 75%, in the range of about 40% to about 60%, or in the range of about 45% to about 55%, the microparticles having an average particle diameter that is less than 50 μm.
15. The composition of the previous item, wherein the composition is in the form colloidal suspension in an aqueous solution.
16. The composition of the previous item, wherein the composition is in the form colloidal suspension in water or a physiological body fluid.
17. The composition of item 15, wherein the composition is in the form colloidal suspension in a solution that comprises a mixture of water and a water-miscible organic solvent, such as a primary alcohol.
18. The composition of item 15, wherein the composition is in the form of colloidal suspension in a mixture of ethanol and water.
19. The composition of the previous item, comprising physically dispersed microparticles suspended in the aqueous solution to obtain a colloidal gel-like matrix comprising the microparticles suspended in the matrix.
20. The composition according to any one of the previous items 16-19, wherein the aqueous solution has a pH in the range of about 7 to about 8, preferably about 7.3 to about 7.5, more preferably about 7.4.
21. The composition according to any one of the previous items 15-20, wherein the aqueous solution comprises a physiological body fluid or other suitable fluid, e.g. saline solution.
22. The composition of any one of the preceding items 14-21, wherein the microparticles have an average particle diameter in the range of about 5 μm to about 50 μm, preferably in the range of about 5 μm to about 25 μm, more preferably in the range of about 5 μm to about 15 μm.
23. The composition of any one of the preceding items 14-22, wherein the microparticles have an average particle diameter that is about 10 μm.
24. The composition of any one of the preceding items 14-23, wherein the partially deacetylated chitosan has a degree of deacetylation that is about 50%.
25. A surgical implant or part thereof, characterised in that it is coated on its outer surface or a part thereof with a surface coating comprising non-crosslinked partially deacetylated chitosan (PDC) having a degree of deacetylation in the range of about 30-75%.
26. The surgical implant or part thereof according to item 25, wherein the degree of deacetylation is in the range of about 30%-75%, in the range of about 35%-60%, in the range of about 40%-60%, in the range of about 45-55%, or about 50%.
27. The surgical implant or part thereof according to item 25 or 26, wherein the chitosan coating has a dry thickness in the range of 2-25 μm, preferably in the range of 5-15 μm.
28. The surgical implant or part thereof according to item 25 or 26, wherein the coating has a wet thickness in the range of 20 to 250 μm, in the range of 50 to 250 μm, in the range of 30 to 150 μm or in the range of 40 to 100 μm.
29. The surgical implant or part thereof according to any one of the items 25-28, wherein the chitosan-based coating is obtainable by immersing the implant in a non-acidified solution comprising the PDC, with a pH above 6, preferably in the range of 7-8, more preferably in range of 7.3-7.5, most preferably about 7.4.
30. The surgical implant or part thereof according to any one of the items 25-29, wherein the chitosan-based coating is obtainable by immersing the implant in an non-acidified colloidal solution or gel comprising the PDC.
31. The surgical implant or part thereof according to any one of the items 25-30, wherein the chitosan-based coating is obtainable by immersing the implant in an non-acidified gel comprising dispersed microparticle PDC.
32. The surgical implant or part thereof according to any one of the items 25-31, wherein the coating comprises microparticle PDC having a mean particle size less than about 50 μm.
33. The surgical implant or part thereof according to any one of the items 25-32 wherein the microparticles have an average particle diameter in the range of about 5 μm to about 50 μm, preferably in the range of about 5 μm to about 25 μm, more preferably in the range of about 5 μm to about 15 μm.
34. The surgical implant or part thereof according to any one of the items 29-33, wherein the colloidal solution or gel or suspension comprises in the range of 0.1% to 20% PDC, or 0.5 to 10% PDC, or preferably in the range from about 1% to about 5% PDC.
35. The surgical implant or part thereof according to any one of the items 25-34, wherein the density of N-acetylglucosamine (NAG) on the surface of the implant, or part thereof, is in range of 0.01-15 mg/cm², or 0.3-5 mg/cm², preferably 0.5-1.5 mg/cm².
36. The surgical implant or part thereof according to any one of the items 25-35, wherein the chitosan coating is non-homogeneous, coating about 10 to about 90% and preferably in the range of about 25% to about 75%, of the outer surface of a unit area of the implant, or part thereof.
37. The surgical implant or part thereof according to any one of the items 25-36, wherein the chitosan coating has a non-homogeneous coating with a coverage in a unit area of 1-5 mm² that is in the range from 10%-90% and preferably in the range of about 25% to about 75%.
38. The surgical implant or part thereof according to any one of the items 25-37, wherein the implant or part thereof is made from one or more material selected from hydroxyapatite, bioglass, titanium or a titanium alloy.
39. The surgical implant or part thereof according to the previous item 38, wherein the surface of the implant has been pre-treated through oxidation by acid treatment.
40. The surgical implant or part thereof according to item 38, wherein the surface of the implant has been pre-treated by washing with an alkali solution.
41. A method of coating a surface of a surgical implant with non-crosslinked partially deacetylated chitosan (PDC), comprising immersing the implant surface in an aqueous solution or gel comprising PDC having a degree of deacetylation in the range of about 30-65%, removing the implant surface from the solution, and allowing the treated surface to dry.
42. The method according to item 41, wherein the PDC has a degree of deacetylation in the range of about 35% to about 60%, preferably about 40% to about 60%, more preferably about 45% to about 55%, even more preferably about 50%.
43. The method according to item 41 or 42, wherein the aqueous solution has a pH above about pH 6 and preferably in the range of about 7.2 to about 7.5.
44. The method of the previous item, wherein the composition is in the form colloidal suspension in water or a physiological body fluid.
45. The method of item 41, wherein the composition is in the form of a colloidal suspension in a solution that comprises a mixture of water and a water-miscible organic solvent, such as a primary alcohol.
46. The method of item 45, wherein the composition is in the form colloidal suspension in a mixture of ethanol and water.
47. The method according to according to any one of the items 41-46, wherein the drying the treated surface comprises incubation at a temperature in the range of 20-80° C.
48. The method according to any one of the items 41-47, wherein the coated surface has a surface coating distribution that is in the range of about 10 to about 100%
49. The method according to any one of the items 41-47, wherein the coated surface has a surface coating distribution that is in the range of about 10 to about 90% and preferably in the range of about 25% to about 75%.
50. The method according to any one of the items 41-49, further comprising pre-treatment of said surface prior to the immersing, where said pre-treatment comprises oxidation by acid treatment, followed by washing in alkali and optional rinsing to neutralise the solution.
51. The method according to any one of the items 41-50, wherein the method comprises at least two coating cycles, wherein each cycle comprises immersing the implant surface in an aqueous solution or gel comprising PDC having a degree of deacetylation in the range of about 30-65%, removing the implant surface from the solution, and allowing the treated surface to dry, and wherein the aqueous solution or gel comprising PDC is different in the respective coating cycles.
52. The method according to the previous item, wherein the first coating cycle comprises immersing the implant surface in an aqueous solution comprising PDC so as to obtain essentially complete coating of the surface, and wherein the second coating cycle comprises immersing the implant surface in an aqueous gel or suspension to obtain a second coating layer on the surface.

As used herein, including in the items, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and items, the terms “comprise”, “including”, “having”, and “contain” and their variations should be understood as meaning “including but not limited to”, and are not intended to exclude other components.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent items that refer to independent items that describe features with “at least one” have the same meaning, both when the feature is referred to as “the” and “the at least one”.

It will be appreciated that variations to the foregoing embodiments of the invention can be made while still falling within the scope of the invention can be made while still falling within scope of the invention. Features disclosed in the specification, unless stated otherwise, can be replaced by alternative features serving the same, equivalent or similar purpose. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so itemed. Any steps described in the specification may be performed in any order or simultaneously, unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination.

The invention is further described by the following non-limiting examples.

### EXAMPLES

### Example 1. Partially Deacetylated Chitosan (PDC)

The production of the PDC comprises a series of processes. It was first deacetylated in concentrated sodium hydroxide to deacetylate chitin into a ca. 50% deacetylated material at a low temperature, e.g. at 60° C. and below, to minimize the formation of blocked distribution of NAG to minimize the crystalline properties of the material. This is crucial, as more clustered NAG will increase the chain to chain interactions and this interferes the chain separation upon protonation of the polymers, and thus its solubility. Moreover, this clustered NAG will increase the interactions forces among chains upon drying of the material, resulting a less amorphous material.

This is followed by the removal of the alkali through a thorough washing process and the recovery of the material. Then the material was dissolved in acid (e.g. citric acid) to facilitate the removal of all insoluble materials in the acidic fluid, through a series of filtration After that the solution was neutralized by addition of NaOH, the polymers were precipitated by adding salt to the fluid. Then the obtained chitosan polymers were collected and thoroughly washed; the purified partially deacetylated chitosan (PDC) material was recovered and dried. Finally, the material packed into a bag and sealed.

This PDC prepared in this manner possesses many different features from classical chitosan available on the market:

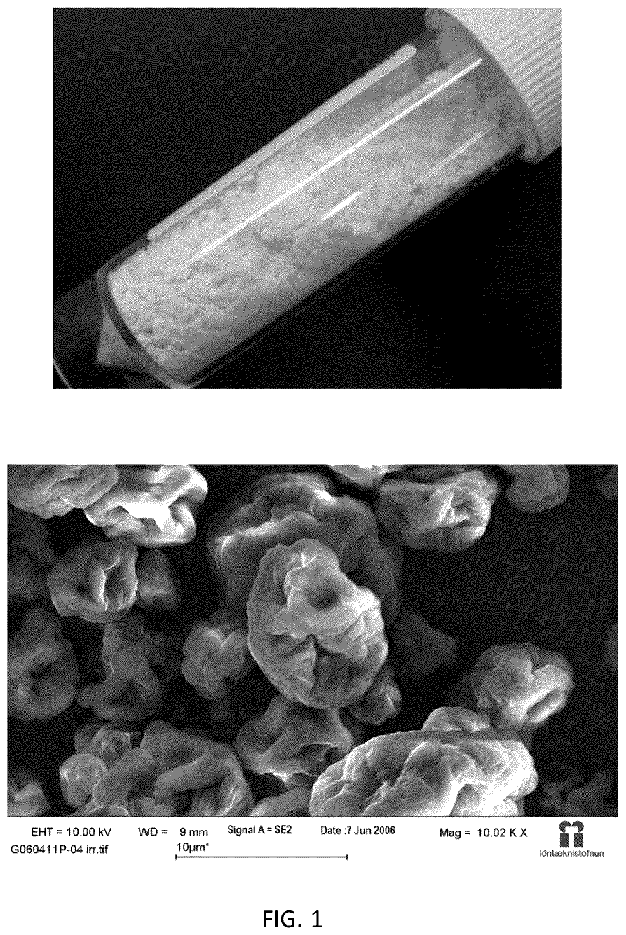

Including, this PDC is highly amorphous and with uniform fine circular particulates of size about 101□□m, as shown in FIG. 1. Contrary to classic chitosan, in which the dissolution of solid chitosan to obtain a chitosan solution could take many hours or even overnight, the dissolution of the PDC obtained by the above method is almost instantaneous. For example, upon dispersing the PDC in a fluid, e.g. in water, the addition of suitable acid (e.g. citric acid or any other suitable organic or inorganic acid) will transform the colloidal fluid into a crystal clear/homogenous solution within seconds. This PDC solution is, as well more resistant to precipitation around physiological pH and beyond than classical chitosan.

This PDC has a near to equal ratio of NAG and GluN, or a more balanced hydrophobic/hydrophilic properties. As a consequence the PDC is able to form a stable mixture with either water-based or most oil-based fluids easily. Moreover, the amorphous and fine particulate nature of the dry PDC enables it to absorb more than 20× of pure water to its weight. All these extraordinary properties render the PDC to challenge more demanding preparation and to open up new fields of applications to meet future biopharmaceutical/medical needs.

The % DD of the PDC can generally be in the range of 45-60% and have a weight-average molecular weight (Mw) of 80-350 kDa. Following table listed a typical example of the common properties of the PDC.

Parameters

| | | | |
|---|---|---|---|
| pH | : | 8.0 | |
| Degree of | : | | |
| Deacetylation | : | 49.9 | % |
| Weighted Average MW (Mw) | : | 300000 | Da |
| Solubility | : | 100 | % |
| Dry Matter Content | : | 93 | % |
| Ash | : | 0.3 | % |
| Proteins | : | — | % |
| Turbidity | : | <10 | NTU |
| Feature | : | Dried powder | |
| Appearance | : | White powder | |
| Particle size | : | <10 | μm |
| Taste and odor | : | Characteristic | |

### Example 2. Coating Method

The titanium plate used in this experiment was Ti ASTM B265 G2, and was cut into 2 cm×1 cm in size. The titanium plate was treated with ION sodium hydroxide for 24 h at 60° C. bath before coating. Then the plate was washed free of alkali, dried and individual plate weight recorded. The designated concentration of PDC was 0.75% (w/v, in 1% acetic acid). The theoretical load amount was 0.655 mg/cm² of PDC or load volume of 0.75% PDC solution at 87.5 μL/cm²

To examine the swelling index, all film-coated titanium plates were soaked into a pool of deionized (DI) water for 2 h, the weight before and after soaking was recorded. The swelling index was determined as the percent ratio of the net wet film weight divided by the net dry film weight.

### Example 3. Analysis of Coating

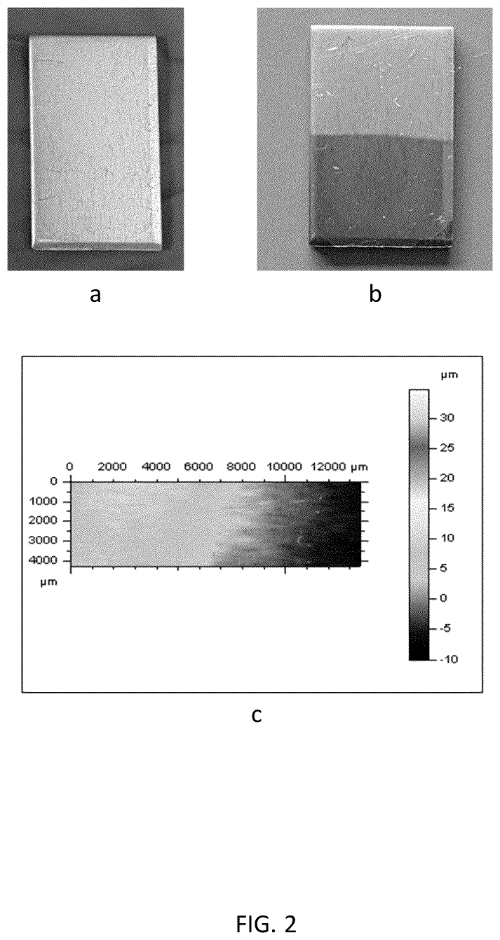

A film thickness measurement was developed by using the profilometry method (Solarius Profilometer). To do so, half of the plate was gold-coated to block light source from penetrating through the transparent film during measurement (FIG. 2(b)). The difference between the height of the gold-coated and uncoated region is the thickness of dry film (FIG. 2(c)). By this measurement, the average coated film thickness for this respective loading concentration and loading amount was found to be 11±2 μm.

### Example 4. Determination of Surface Roughness

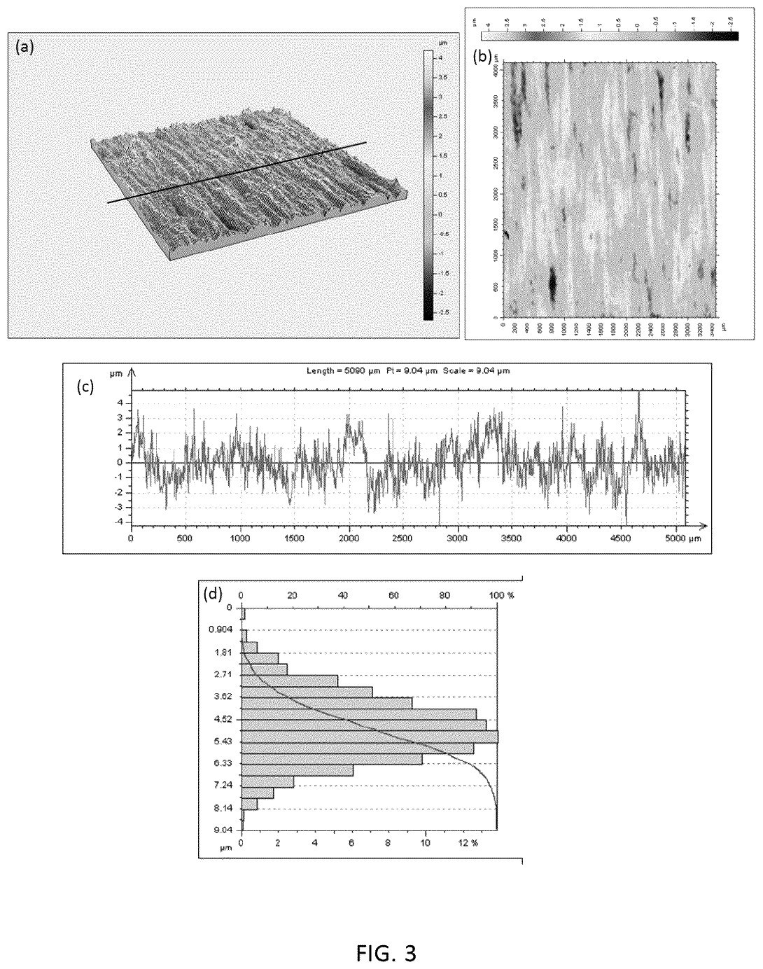

Surface topography of PDC coated titanium at dry state was measured by a profilometer as shown in FIG. 3. In (a) is shown a 3D view of the surface roughness of a typical titanium plate, coated with 1% PDC (w/v). (b) Topography image of the coated surface in 2D view. (c) Actual recorded surface roughness along the line indicated in FIG. 3(a). (d) Distribution of surface roughness along the line as shown in FIG. 3(a).

As can be seen, the PDC coating provides a smooth and homogeneous coat across the titanium surface.

### Example 5. Swelling Properties of PDC Films

The swelling properties of PDC-coated surfaces were determined. This was done by measuring the swelling Index of coated PDC, before and after 4 weeks of soaking in water.

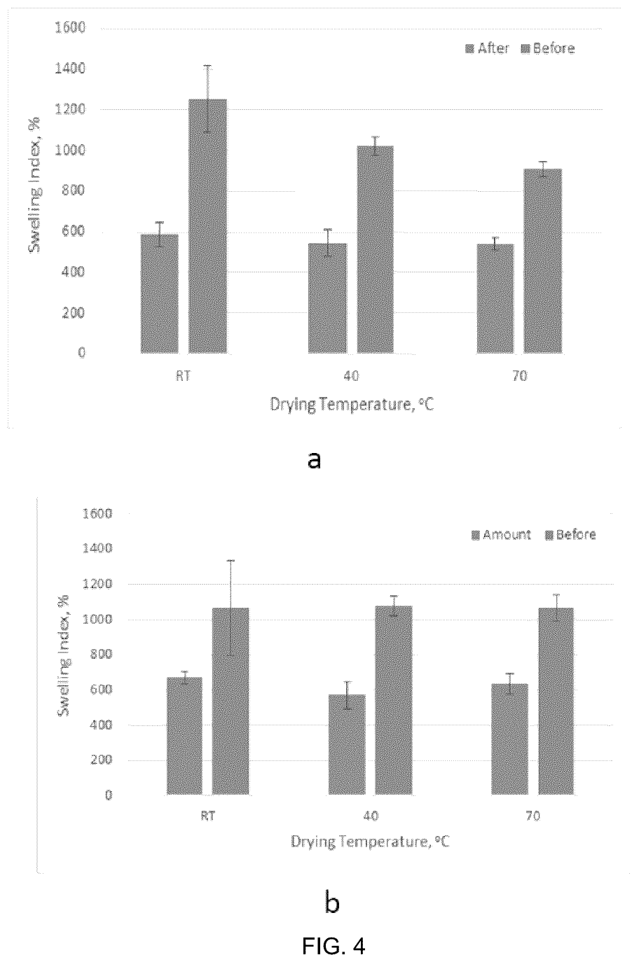

The film was prepared with 0.75% PDC solution in 1% acetic acid in water only or mixture of athanol/water. Results are shown in FIG. 4. In (a), the effect of temperature when water only is used to prepare the PDC solution, while in (b) the swelling is shown when ethanol:water, at ratio of 1:1, is used to prepare the PDC solution.

There is a significant difference in swelling Index among the treatments before and after soaking for 4 weeks (p<0.05), in that the swelling index is significantly reduced upon soaking. There is also a significant difference as a function of drying temperature, with higher temperature leading to lower swelling index.

When ethanol:water (1:1) is used to prepare the PDC solution (FIG. 4b), there is a significant difference in swelling Index among the treatments before and after soaking for 4 weeks (p<0.05). However, the drying temperature does not produce a significant impact on the swelling index of the film.

### Example 6. Control of Swelling Index

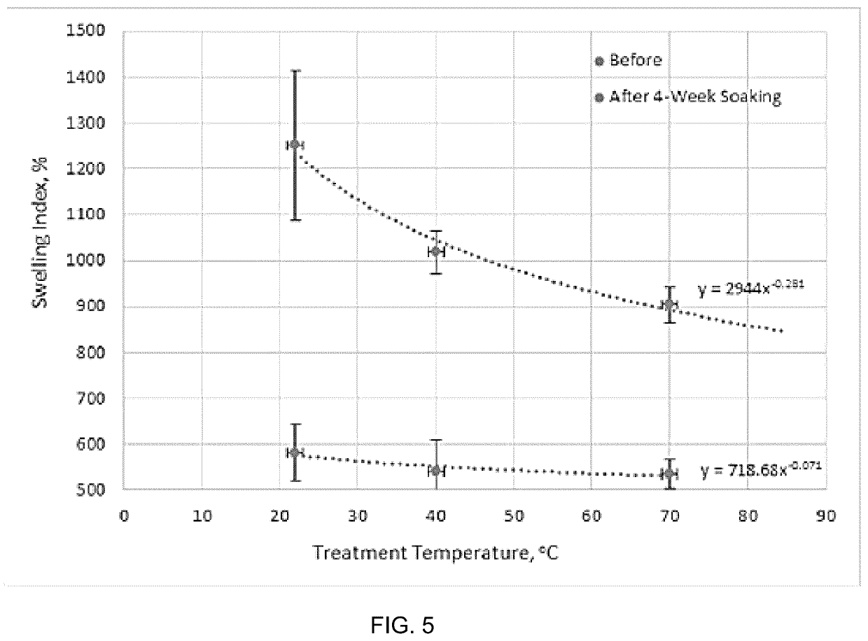

The relationship of drying temperature and swelling behaviour of PDC films in water was investigated, as shown in FIG. 5. Results indicate that the initial thickness of the film is controllable by varying the temperature of the drying step.

### Example 7. Coating Surface Determination by SEM

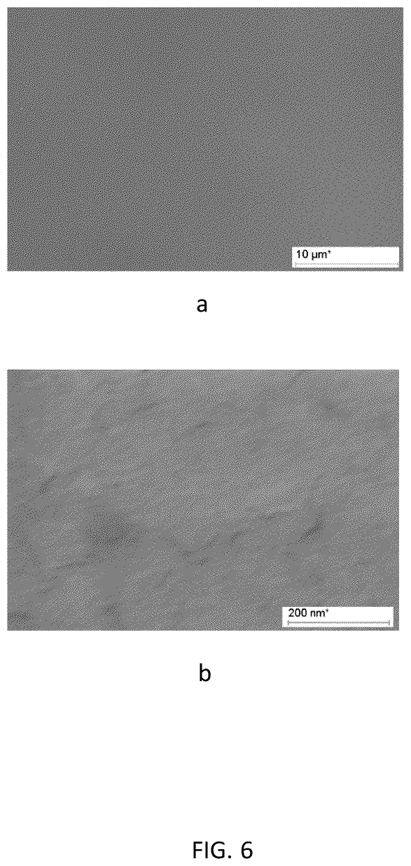

Scanning Electrode Microscopy (SEM) was used to study the coating of treated surfaces. In FIG. 6, typical Scanning Electrode Microscopy (SEM) images of titanium plate coated with PDC dissolved in acetic acid is shown. (a) 2000× magnification, (b) 100,000× magnification. As can be seen, there is a nearly perfect uniform coating of the titanium surface.

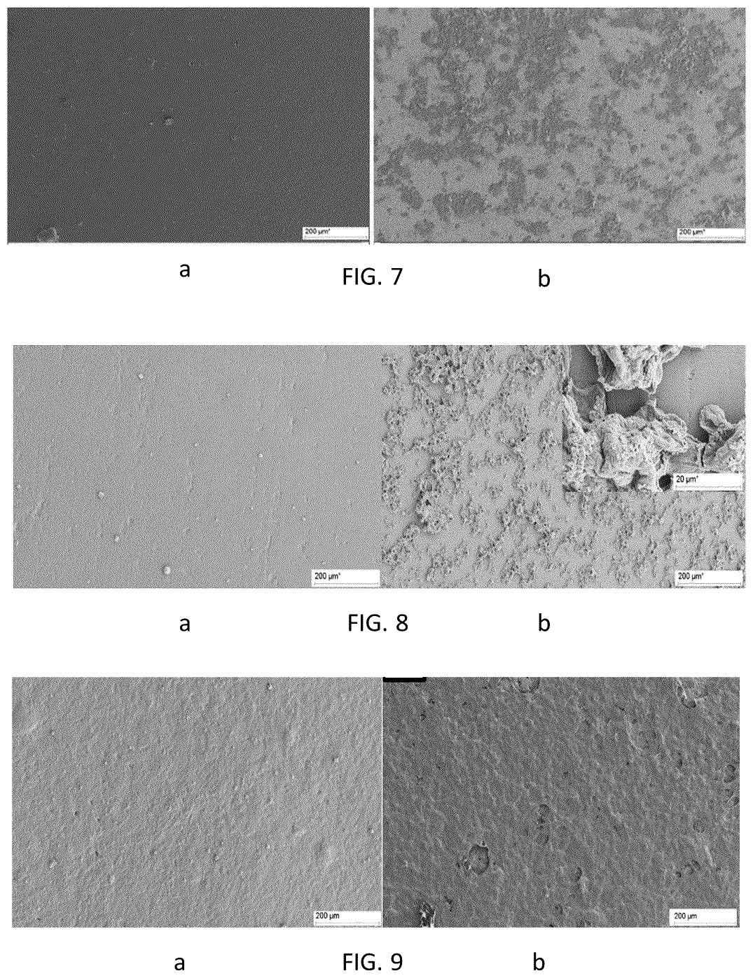

The coating obtained by different methods and its coverage over the coated surface was also determined, as illustrated in FIGS. 7-9. In FIG. 7 there is shown an example of PDC coating on a titanium substrate. (a) the coating obtained by soaking in a fully dissolved PDC solution (true solution) and (b) coating obtained by soaking in colloidal PDC. Surface coating using a true solution is nearly complete (greater than 99%), while the coating using the colloidal method is non-homogenous, with about 41% of the surface area coated.

Results for coated Bioglass and Hydroxyapatite surfaces are shown in FIGS. 8 and 9 respectively, with numerical results shown below. For all surfaces, essentially complete coating (greater than 99%) is obtained using fully dissolved PDC, while treatment with colloidal PDC results in about 50% coating coverage.

| | Area Coverage, % | |
|---|---|---|
| | True solution | Colloidal |
| Substrates | Method | Method |
| Titanium | 99+ | 41 |
| Bioglass | 99+ | 52 |
| Hydroxyapatite | 99+ | 83 |

### Example 8. Film Material Loss after Prolonged Soaking in Water

To investigate the effects of the fluids, i.e. with water only and with ethanol/water mixture, used for the preparation of PDC solution and prolonged incubation of treated surfaces in an aqueous environment, treated surfaces were incubated for 4 weeks in water, and the loss of surface coating material was determined.

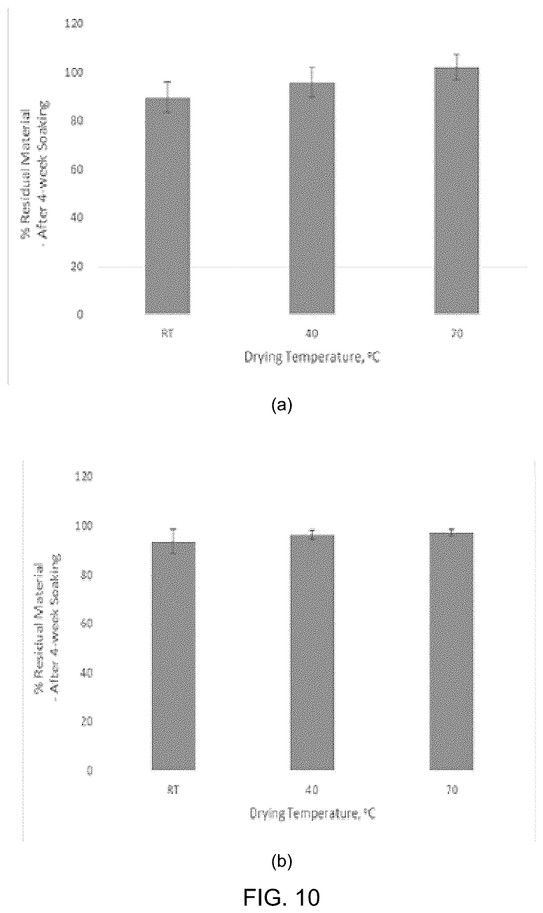

As can be seen in FIG. 10, there is a difference in net dry weight of films among the water-only aqueous system (FIG. 10(a); p<0.05), but not the films prepared by solvent/water aqueous system (ethanol:water 1:1; FIG. 10(b); p>0.05). There is a maximal loss of 10% material at max after 4-week soaking in water (RT drying).

However, the majority of films were found to be intact, indicating that the coating method results in a reliable film for coating purposes.

### Example 9. Determination of Surface Distribution by SEM Upon Treatment with PDC in PBS Solution

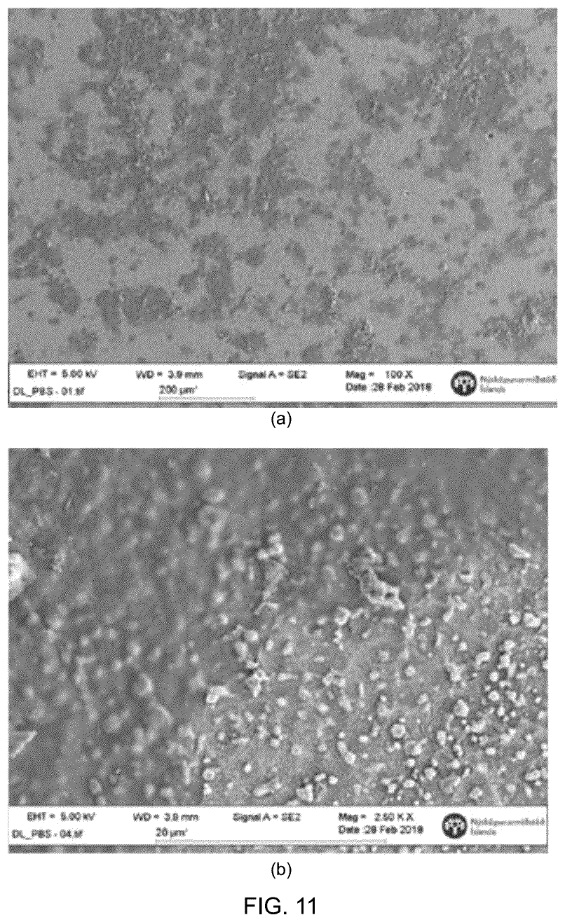

The effect of physiological buffer on the surface distribution of PDC coating was determined by SEM. Titanium coated with PDC dispersed in PBS (phosphate-buffered saline) solution. The subsequently obtained coating was investigated by SEM, as shown in FIG. 11. In (a) coated PDC (darker grey regions) can be seen to have an organic distribution on the surface of titanium at a magnification of 100×; In (b) the roughness at 2500× magnification can be seen, with an uneven thickness of the coating on the surface. The relatively rough surface facilitates cell adhesion, and thus coating obtained by this method is suitable for in vivo use.

### Example 10. AFM on Coating of PDC in Acetic Acid Solution and Dispersed with PBS Solution

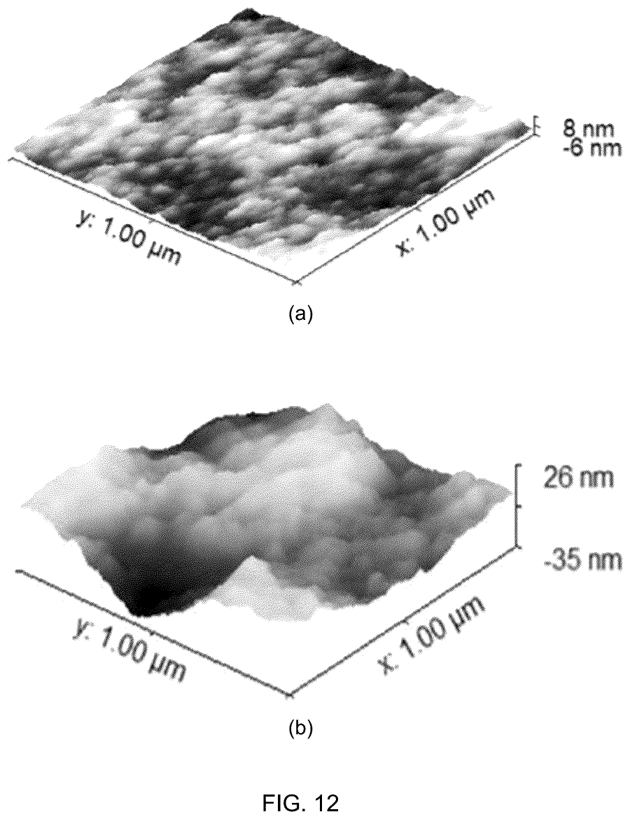

Atomic Force Microscopy (AFM) was used to study PDC coating of dried coated films obtained by different coating methods, i.e. by homogeneous true solution coating (PDC fully dissolved in acetic acid) and by non-homogeneous coating of PDC dispersed in PBS buffer. Results are shown in FIG. 12. (a) chitosan fully dissolved in acid and coated onto titanium by homogeneous method; and (b) PDC dispersed in PBS solution and coated onto titanium by the non-homogeneous method. As can be seen, the non-homogeneous method produces a coating with larger variation in both the thickness and roughness of the film.

1. A chitosan-based coating for a surgical implant, the coating comprising non-crosslinked partially deacetylated chitosan (PDC) having a degree of deacetylation in the range of about 30-75%, wherein the PDC is in the form of microparticles having an average particle size of less than 50 μm.

2. The chitosan-based coating of claim 1, wherein the microparticles have a spherical shape.

3. The chitosan-based coating of claim 1, wherein the PDC is amorphous.

4. The chitosan-based coating of claim 1, wherein the PDC has a degree of deacetylation in the range of about 35% to about 70%, in the range of about 35% to about 60%, in the range of about 40% to about 75%, in the range of about 40% to about 60%, or in the range of about 45% to about 55%.

5. The chitosan-based coating according to claim 4, wherein the coating has a dry thickness in the range of 2-25 μm, preferably in the range of 5-15 μm, or wherein the coating has a wet thickness in the range of 20 to 250 μm, preferably in the range of 40 to 100 μm.

6. The chitosan-based coating according to claim 1, wherein the coating has a surface roughness that is in the range of about 25-500 μm, in the range of 5-250 μm, in the range of about 20-100 μm, in the range of about 25-100 μm, in the range of about 5-100 μm or in the range of 5-50 μm.

7. The chitosan-based coating according to claim 1, wherein the coating comprises a non-homogeneous layer with a coverage over a unit area on a surgical implant that is in the range of about 10 to about 90% and preferably in the range of about 25% to about 75%.

8. The chitosan-based coating according to claim 1, wherein the coating comprises PDC in the form of microparticles with an average particle size of less than 30 μm or less than 10 μm.

9. A composition for coating a surgical implant, the composition comprising microparticles containing partially deacetylated chitosan (PDC) with a degree of deacetylation in the range of about 45% to about 55%, the microparticles having an average particle diameter that is less than 50 μm.

10. The composition of claim 9, wherein the composition is in the form of a colloidal suspension in an aqueous solution.

11. The composition of claim 9, wherein the PDC is amorphous.

12. The composition of claim 11, comprising physically dispersed microparticles suspended in the aqueous solution to obtain a colloidal gel-like matrix comprising the microparticles suspended in the matrix.

13. (canceled)

14. A surgical implant or part thereof, characterised in that it is coated on its outer surface or a part thereof with a coating according to claim 1.

15. The surgical implant or part thereof of claim 14, wherein the PDC has a degree of deacetylation in the range of about 35%-60%, in the range of about 40%-60%, in the range of about 45-55%, or about 50%.

16. (canceled)

17. The surgical implant or part thereof according to claim 14, wherein the surface coating has a dry thickness in the range of 2-25 μm, preferably in the range of 5-15 μm.

18. The surgical implant or part thereof according to claim 14, wherein the surface coating has a wet thickness in the range of 20 to 250 μm, in the range of 50 to 250 μm, in the range of 30 to 150 μm or in the range of 40 to 100 μm.

19. The surgical implant or part thereof according to claim 14, wherein the surface coating is obtainable by immersing the implant in an non-acidified colloidal solution or gel comprising the PDC.

20. (canceled)

21. The surgical implant or part thereof according to claim 14, wherein a density of N-acetylglucosamine (NAG) on the surface of the implant, or part thereof, is in range of 0.01-15 mg/cm², or 0.3-5 mg/cm², preferably 0.5-1.5 mg/cm².

22. The surgical implant or part thereof according to claim 14, wherein the chitosan coating is non-homogeneous, coating about 10 to about 90% and preferably in the range of about 25% to about 75%, of the outer surface of a unit area of the implant, or part thereof.

23. A method of coating a surface of a surgical implant with non-crosslinked partially deacetylated chitosan (PDC), comprising immersing the implant surface in an aqueous solution or gel comprising PDC having a degree of deacetylation in the range of about 30-65%, wherein the PDC is in the form colloidal suspension in water or a physiological body fluid, removing the implant surface from the solution, and allowing the treated surface to dry.

24. The method of claim 23, wherein the composition is in the form of a colloidal suspension in a solution that comprises a mixture of water and a water-miscible organic solvent, such as a primary alcohol.

25. The method of claim 23, wherein the PDC is amorphous.

26. The method according to claim 23, wherein the method comprises at least two coating cycles, wherein each cycle comprises immersing the implant surface in an aqueous solution or gel comprising PDC having a degree of deacetylation in the range of about 30-65%, removing the implant surface from the solution, and allowing the treated surface to dry, and wherein the aqueous solution or gel comprising PDC is different in the respective coating cycles.

27. The method according to claim 26, wherein the first coating cycle comprises immersing the implant surface in an aqueous solution comprising PDC so as to obtain essentially complete coating of the surface, and wherein the second coating cycle comprises immersing the implant surface in an aqueous gel or suspension to obtain a second coating layer on the surface.

28. The method of claim 23, wherein the surface consists of a material selected from stainless steel, titanium, titanium alloy, cobalt chrome alloy, bioglass, hydroxyapatite/calcium phosphates or mixtures thereof.
